(19) United States
(12) Patent Application Publication
(10) Pub. No.: US 2007/0172945 A1
(43) Pub. Date: Jul. 26, 2007

(54) Bioreactor

(76) Inventors: Ronan Desmond O'Kennedy, Kent (GB); Peter Leonard Steward, Kent (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 11/568,580

(22) PCT Filed: May 4, 2005
(86) PCT No.: PCT/GB05/01683

§ 371 Date: Nov. 2, 2006

(30) Foreign Application Priority Data
May 6, 2004 (GB) 0410118.4

(51) Int. Cl. C12N 5/00; C12N 1/20; C12M 3/00

(52) U.S. Cl. 435/325; 435/252.1; 435/295.2

(57) ABSTRACT

The present invention concerns a plunging jet bioreactor (1) comprising a mechanism (4), (5) adapted in use to form culture fluid into a hollow flow stream and to introduce into the hollow of the flow stream an oxygen-containing gas. In some embodiments the mechanism comprises concentrically arranged outer (4) and inner tubes (5) wherein the inner tube is in flow communication with the culture fluid container whereby the culture fluid flows over the inner tube to form the hollow flow stream into which the oxygen from the inner tube is introduced.

### FIELD OF THE INVENTION

The present invention relates to the field of culturing micro-organisms and cells such as eucaryotic cells. More particularly, it relates to a bioreactor apparatus for culturing micro-organisms and/or cells such as eukaryotic cells and to methods for culturing micro-organisms and/or cells such as eukaryotic cells using the bioreactor apparatus as described herein.

### BACKGROUND

There is increasing demand for the production of biopharmaceutical products from microbial, yeast, mammalian or plant cell culture. These products may include recombinant and non-recombinant peptides and proteins, recombinant plasmid DNA for genetic vaccination or gene therapy applications.

Production of biopharmaceuticals generally requires the construction of a producing cell line and subsequent culture of that cell line to elicit expression of the required product. Biopharmaceutical products destined for use in humans or animals are subject to regulatory authority manufacturing controls and must be manufactured under good manufacturing practice (GMP) conditions. To establish and maintain manufacture under these GMP conditions, biopharmaceutical organisations design, install, validate and maintain plants that are dedicated to manufacture of biopharmaceutical products under GMP conditions. The product material used in the initial testing stages of commercialisation is also made under GMP conditions but plants are used to successively produce multiple products. Before proceeding with the manufacture of a new product, the manufacturer must demonstrate to the regulatory authorities that the plant has been cleaned down correctly and no detectable traces of the previous product exists on any product contact surfaces. The additional regulatory requirements of equipment validation and plant clean down between products imposes a time burden and cost on the efficient operation of a manufacturing plant. This plant cleandown and associated confirmation of cleandown by analytical testing adds a significant time to the production of the final product. Indeed in some instances, the clean-down and re-validation analysis may take longer than the culturing process in the first place. Thus techniques and/or equipment that promote efficient production of biopharmaceutical products are required.

There is a need to produce microorganisms or cells in a bioreactor where the cells have been engineered to produce a bio-molecule of pharmaceutical interest. Large scale production of engineered cell lines is generally carried out in bioreactor systems that maintain and control physiological parameters and ensure optimum growth. A primary design feature of bioreactors is to provide the necessary mixing gas transfer in the vessel. As cells grow, they use up oxygen and produce carbon dioxide. The bioreactor must be designed to ensure efficient transfer of oxygen into the culture whilst allowing efficient removal of carbon dioxide. This is generally achieved in a stirred tank bioreactor that is sparged with a compressed gas containing oxygen. Some alternative bioreactor mixing systems are also possible. These alternative systems can, to some extent, achieve sufficient gas mixing to allow growth of slow growing cultures such as mammalian cells but are not sufficient to support more intensive microbial growth. Table 1 compares oxygen transfer rates of different systems to conventional stirred tank bioreactors used for culturing mammalian and microbial cells. One such system is the commercially available single use bioreactor, where mixing and gas exchange is induced by inducing a wave-type liquid using a rocking motion (Singh, 1999 Cytotechnology 30:pp 149-158). This single use bioreactor operates within the gas mixing range for use with mammalian cell culture but may not be able to provide sufficient gas mixing required for the higher oxygen requirements of microbial cultures. Plunging jets are also known in waste water treatment and bioreactors have been described in relation to the field of “tissue engineering” (for example U.S. Pat. No. 6,670,169 B1 Schob et. Al). Novais et al (Novais, Tichener-Hooker and Hoare, 2001, Biotech. Bioeng 75:143-153) suggested using a single plunging jet in a disposable bag format but no other details were given. Novais et al suggested that this arrangement would result in reduced bioproduct yields due to insufficient oxygen transfer.

An alternative mixing regime using a jet outlet has been demonstrated by Zaidi et al (Zaidi, Ghosh, Schupme & Deckwer, 1991, Appl. Microbiol. Biotechnol., 35:330-333).

The tip of the jet is surrounded by a ring with inward pointing holes from which streams of oxygen-containing gas are blown onto the liquid stream exiting the jet.

TABLE 1
Range of K_{L}a for different reactor configurations

| Reactor type | K_{L}a (min) |
|---|---|
| Conventional Stirred tank bioreactor | |
| (Mammalian) | |
| Small Scale 3L | 0.0005 to 0.007 |
| Pilot scale 150L | 0.0003 to 0.003 |
| (in-house data) | |
| Conventional Stirred tank bioreactor | |
| (Microbial) | |
| (Doran et al 1997) | 0.02 to 0.25 |
| (50L Applikon) | 0.05 to 0.4 |
| “Wave” Disposable Bioreactor | 0.00032 to 0.001 |
| Singh et al (1999) | |
| Plunging Jet | 0.01 to 0.2 |
| (Zaidi et al 1991) | |

### SUMMARY OF THE INVENTION

The present invention provides a bioreactor apparatus for culturing cells such as eukaryotic cells, plant and yeast or microorganisms that is easy to use, inexpensive and versatile. It enables cells and microorganisms to be grown safely. It is an aim of the present invention to improve gas exchange to a level where cell lines used for production of biologically relevant therapeutics such as peptides, proteins, plasmid DNA, viruses and phage may be cultured more effectively. The invention provides a bioreactor apparatus which provides inlet gases mixed into the core of plunging liquid jets. Such an improved bioreactor provides gas mixing in the jet and at the surface of the liquid resulting in more efficient gas transfer required to support growth in a microbial bioreactor or intensive mammalian cell culture.

Accordingly, the present invention provides a bioreactor apparatus for culturing micro-organisms and/or cells in a culture fluid comprising a culturing container for the culture fluid and a circulation system to circulate the culture fluid out of, and back into, the culturing container, wherein the circulation system has a mechanism adapted in use to form the culture fluid into a hollow flow stream and to introduce an oxygen-containing gas stream into the hollow of the flow stream of the culture fluid.

In some embodiments there is provided a bioreactor apparatus for culturing micro-organisms and/or cells in a culture fluid said apparatus comprising a culturing container comprising the culture fluid and micro-organisms and/or cells and a circulation system to circulate the culture fluid out of, and back into, the culturing container, wherein the circulation system has a mechanism adapted in use to form the culture fluid into a hollow flow stream and to introduce an oxygen-containing gas stream into the hollow of the flow stream of the culture fluid.

It will be appreciated that the hollow flow stream may encompass one or more oxygen-containing gas streams, that is, there are a plurality of hollows into which the oxygen-containing gas stream can be introduced.

In particular, the mechanism is adapted in use to form the culture fluid into an annular flow stream having a hollow core and to introduce an oxygen-containing gas stream into the hollow core of the annular flow stream of the culture fluid.

In one embodiment, the mechanism of the circulation system comprises at least one pair of inner- and outer-arranged tubes, the inner tube of at least one pair being in flow communication with a supply of the oxygen-containing gas and the outer tube being in flow communication with the culturing container whereby the culture fluid is able to flow in the outer tube over the inner tube to form the hollow flow stream into the hollow of which the oxygen-containing gas is able to be introduced via the inner tube. In particular, the inner and outer tubes may be concentrically arranged. In some embodiments, the apparatus of the invention has a venturi ratio of between 0.2 to 0.8 inclusively, preferably 0.5 or greater, e.g. 0.6., 0.7, 0.8. Venturi ratio is the ratio of distance of the venturi nozzle (i.e. gas nozzle) is between the liquid entry point and the exit point of the liquid jet and may be defined mathematically as:

JL−AD=distance between liquid entry and liquid jet exit

TTL=Distance between gas entry point and liquid entry point

$$Venturi ratio = \frac{TTL}{( JL - AD )}$$

Thus in some embodiments, the apparatus comprises at least one pair, preferably between two and four pairs of concentrically arranged outer and inner tubes, the inner tube of at least one pair being in flow communication with a supply of the oxygen-containing gas and the outer tube being in flow communication with the culturing container whereby the culture fluid is able to flow in the outer tube over the inner tube to form the hollow flow stream into the hollow of which the oxygen-containing gas is able to be introduced via the inner tube wherein the apparatus has a venturi ratio of between 0.2 to 0.8, preferably 0.5 or greater, e.g. 0.6, 0.7, 0.8. The principle is illustrated in FIG. 3.

In some embodiments, the liquid jet velocity is between 1.5 meters/sec to 20 meters/second depending on scale. In typical embodiments gas flow rates are generally calculated relative to the vessel working volume (vvm=volumes per volume per minute). Thus in some embodiments there is a vvm of 0.25 to 2.25.

In a further embodiment the circulation system has at least one efflux nozzle with an outlet located above the liquid culture surface and oriented into the container for delivering the culture fluid back into the culturing container. In some embodiments, the efflux nozzle is configured to deliver the culture fluid back into the container in the form of a jet. In some embodiments, the outlet of the outer tube forms the efflux nozzle.

In a preferred embodiment the mechanism is adapted such that the oxygen-containing gas is entrained into the hollow of the annular flow stream. Alternatively, the outer and inner tubes are arranged such that the oxygen-containing gas (e.g. air) is able to be drawn into the hollow of the flow stream in the outer tube by the venturi effect.

Additional modifications to such a bioreactor apparatus are also the subject matter of the invention such as the number of liquid jets, the jet angle, number of gas nozzles, number of inner tubes within an outer tube to form the efflux nozzle and the bioreactor aspect ratio (liquid depth, width). All of these features can be optimised to improve gas/culture fluid mixing. Thus, in some embodiments, the bioreactor comprises a plurality of liquid jets, preferably between 2 and 4 jets. In other embodiments, the jet angle ( that is the angle of the jet as it contacts the surface of the liquid culture fluid) is orientated at an inclined angle of 70° to 75° or thereabouts to the plane of the culture liquid surface.

It is a further embodiment of the present invention to provide a single use bioreactor apparatus having the features set forth above. The advantages of such a bioreactor include, but are not limited to, a reduction in product turnaround, minimal clean-down of the plant and a reduction in cycle times and analytical resources required. Additional advantages include a reduction in equipment validation.

The culturing container may be constructed from a waterproof semiflexible or flexible material. In particular from polyvinyl chloride, or one or more layers of PVC or PTFE sheets. Additionally, the circulation system may be constructed of waterproof semiflexible or flexible material such as silicon elastomer or platimum treated silicon elastomer.

In one embodiment, the bioreactor apparatus has a pump for pumping the culture fluid out of the container and back thereinto. Suitable pumps include a peristaltic pump in which the pump heads do not come into direct contact with the culture fluid or alternatively a pump with a disposable pump head. Such pumps are well known or apparent to those skilled in the art and are available commercially from suppliers such as Watson Marlow Bredel and Levitech Where shear damage to the cells or microorganisms is a concern a low shear pump maybe used. Where deleterious effects from foaming are a concern then a surfactant such as Pluronic F-68 maybe added to the culture media.

The bioreactor apparatus may further comprise sensors, in particular, single use sensors, in order to sense parameters that characterise the growth environment in which cells are cultured. These include but are not limited to optimisation of the control of temperature, and/or pH and/or dissolved oxygen tension. These sensors may be located either within the container or the circulation system.

Further aspects and features of the present invention are set forth in the exemplary embodiments of the invention which will now be described with reference to the accompanying Figures of drawings.

### BRIEF DESCRIPTION OF THE DRAWINGS

FIG. 1 is a schematic diagram of a disposable microbial bioreactor in accordance with the present invention.

FIG. 2 is a schematic diagram of a 5 L Test bioreactor in accordance with the present invention. Illustrative dimensions are set forth in the description below.

FIG. 3 is a schematic diagram of a Venturi plunging jet design usable in the invention. Illustrative dimensions are set forth in the description below.

FIG. 4 is a summary of results from Example 1 gas mixing response surface design experiment.

FIG. 5 illustrates the K_{L}a response surface predictions for the effect of gas and liquid flow rate at high venturi ratio and jet height.

FIG. 6 illustrates the plunge depth response surface predictions for the effect of gas and liquid flow rate at high venturi ratio and jet height.

FIG. 7 illustrates the effect of 15° jet angle on the K_{L}a response.

FIG. 8 illustrates the effect of a crimped venturi air with two gas outlets on the K_{L}a response.

FIG. 9 shows summary of results from Example 2 gas mixing response surface design experiment.

FIG. 10 illustrates the effects of different venturi ratio on gas mixing parameter (K_{L}a) from Example 2 response surface design experiment.

FIG. 11 illustrates the response surface model prediction of the gas and liquid flow rate settings required to achieve a minimum K_{L}a.

FIG. 12 illustrates a comparison between performance of scaled up venturi plunging jets in comparison to prediction made by the small scale jet model

FIG. 13 illustrates a comparison of growth profiles of E. coli DH1 pXY grown in a conventional stirred tank reactor and a venturi plunging jet reactor. Results are expressed relative to the maximum biomass achieved in the STR control runs (N=2).

FIG. 14 shows agarose gel electrophoresis of samples taken from a conventional stirred tank reactor and a venturi plunging jet reactor. Lanes 1 to 6=STR control runs, 7 to 9 VPJ—No DOT control, 10 to 12 VPJ with DOT control.

FIG. 15 illustrates a densitometric analysis of EtBr agarose gel of plasmid DNA species. Peak areas were not corrected for differential ethidium bromide (EtBr) binding to linear and open circular plasmid DNA.

### DETAILED DESCRIPTION OF THE INVENTION

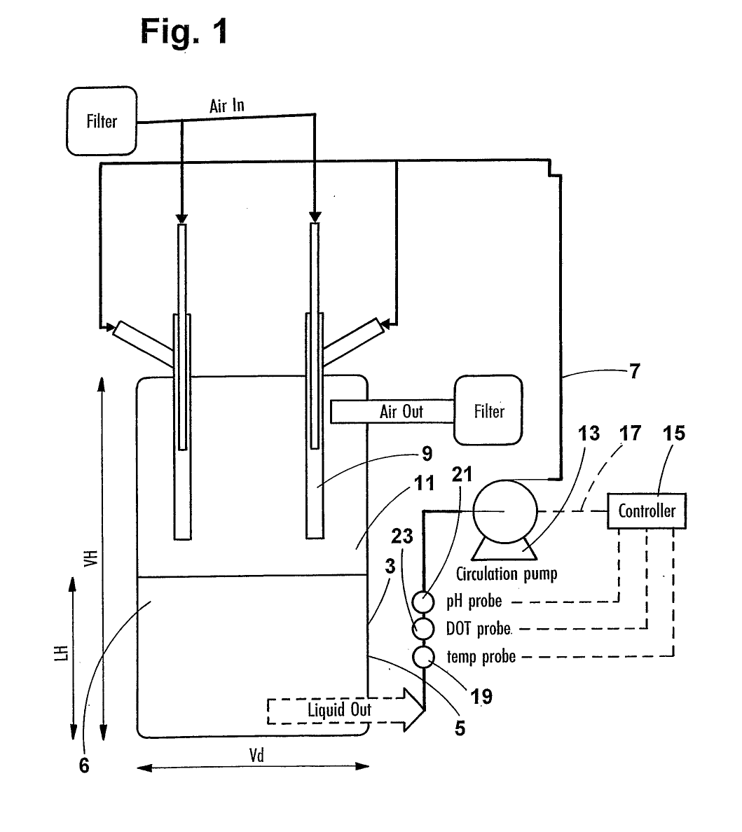

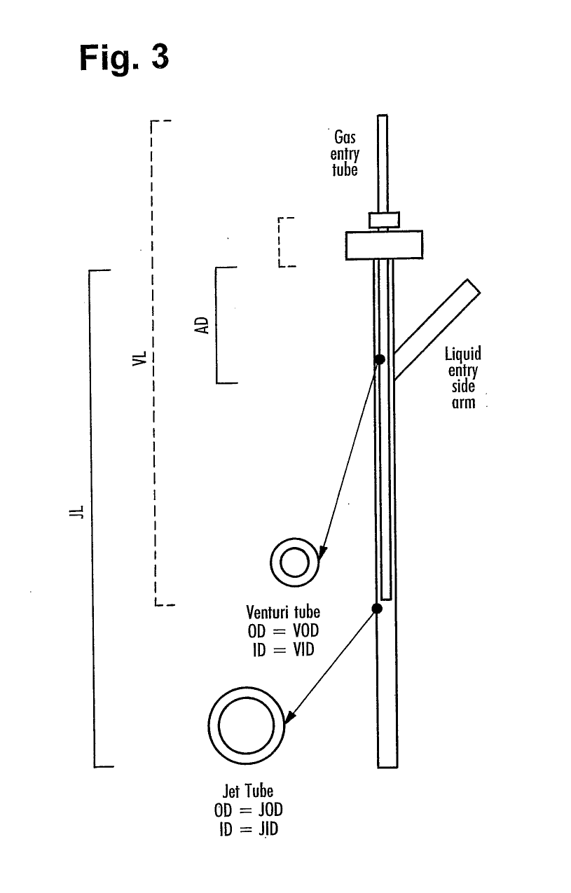

Referring to FIG. 1, there is shown a system of the present invention. The system has a flexible plastic cylindrical or rectangular bag (1) (i.e. “culturing container”) which is supported on all sides by a rigid container (not shown). Culture fluid (2) is drawn from the bottom of the bag using one or more recirculation loops (3) and re-enters the top of the bioreactor apparatus via one or more jet nozzles (4). Jets (4) may be as shown in FIG. 3 and are designed to effect efficient oxygen transfer into the culture medium. Culture liquid flow is injected into the bioreactor apparatus at high velocity through the jet and oxygen containing gas is introduced into the center of the culture liquid flow (5) to effect oxygen gas transfer into the liquid and to induce carbon dioxide gas transfer out of the liquid. Under standard liquid flow conditions, gas is drawn into the liquid stream due to a venturi effect. Gas can also be sparged into the liquid stream at controlled flow rates to induce more turbulent flow in the jet stream. Interchange of oxygen and carbon dioxide gases into and out of the liquid phase also occurs as the liquid jet plunges through the bioreactor headspace (6) and at the surface of the culture fluid. The gas orifice in the jet can be modified to have two or more holes to increase the gas bubble (interfacial) area that comes into contact with the liquid jet. The liquid jet exits the jet nozzle at a sufficient velocity to induce circulation within the bioreactor bag. The tips of the jets can be angled which increases lateral mixing in the culture fluid thus improving mixing in the bioreactor. Inlet gas is introduced into the jets and are expelled from the reactor via inlet and exit gas a sterilizing filters (7).

The jet nozzles (4) of FIG. 1 are shown in greater detail in FIG. 3. Illustrative dimensions thereof are set forth below:

Dimensions

| | JL | VL | AD | VOD | VID | JOD | JID |
|---|---|---|---|---|---|---|---|
| 5 L Scale jet (mm) | 250 | 300 | 35 | 3 | 2.5 | 7.5 | 6.0 |
| 50 L Scale Jet (mm) | 250 | 300 | 25 | 7.5 | 6.0 | 13.5 | 12.0 |

These dimensions apply to the jet nozzle used in the system described in Example 1 below but other dimensions are possible. Referring to FIG. 3, the jet nozzle comprises an inner and outer arranged tube wherein the inner tube is in flow communication with a supply of the oxygen-containing gas and the outer tube is in flow communication with the culture fluid (2). As shown in FIG. 3, culture fluid enters the jet nozzle through the liquid entry side arm and exits through the jet tip. The internal and external diameters of the liquid entry side arm are typically the same as the jet tube. The gas entry tube enters the jet tube from the top and joins the liquid jet flow at the intersection between the liquid side arm and the gas entry tube. As liquid flows past the gas entry tube, an area of low pressure forms at the liquid contact point. This area of low pressure draws gas into the centre of the liquid jet by the venturi effect. Oxygen gas exchange in the jet tube occurs at the interface between the liquid and gas. The area of the gas/liquid interface can be increased by crimping the gas jet in the venturi so that two streams of gas contact the liquid.

Once the culture fluid and gas mixture exit the jet tip, gas exchange occurs both at the core of the jet and on the jet's external surface. Because of this, the mass of gas exchanged will be directly proportional to the distance between the jet tip and the bulk liquid surface. When the jet hits the surface of the bulk liquid, additional gas is entrained in the plunge pool at the liquid surface. The incident angle of the jet on the liquid surface will have a positive effect on gas mixing by increasing the gas entrained in the jet plunge pool and by inducing lateral mixing in the bulk liquid.

Liquid flow to the jets can be driven by use of pumps (8). One or more pumps can be used to drive culture fluid flow in one or more recirculation loops. In one such embodiment, peristaltic pumps, where pump heads do not come into direct contact with the culture fluid, can be used to drive culture liquid flow. Alternatively, a pump with a disposable pump head can be used to drive culture liquid flow.

In addition to these embodiments, dissolved gases, such as oxygen or carbon dioxide, present in the culture fluid can be controlled by modulating the jet flow rate. In this case, the pump is linked to an electronic controller (9) capable of detecting the dissolved gas concentration and applying a control signal to the pump to modulate the flow rate through the loop. The controller can also control the flow rate of gas into the efflux nozzle and modulate the pump flow to cause pulsing in the liquid flow, thus inducing better gas exchange.

The bag (1) may be constructed from a waterproof semiflexible or flexible material, preferably but not limited to poly vinyl chloride. The bag (1) may be constructed of one or more layers of PVC or PTFE sheets. The tubing that carries gas and feed supplies to and from the bioreactor container are constructed from waterproof semiflexible or flexible material such as silicon elastomer, platinum treated silicon elastomer or other material suitable for contact with the culture fluid. Feed and inoculum addition lines may also be constructed of thermoelastic polymer tubing that facilitates the use of thermal tubing welders so that aseptic connections to the bioreactor can be made.

To achieve optimal growth of culture the bioreactor temperature must be controlled within a tight operating range. It is envisaged that the temperature can be sensed using a temperature probe located within the bioreactor apparatus in an indwelling sheath or, as shown in FIG. 1, a temperature probe (10) within the circulation system or as an integral part of the bioreactor container. It is envisaged that the temperature probe is an electrical component (such as a thermocouple or thermosistor) or optical based sensor that uses spectrographic or fluorescence measurements to infer temperature. Its is envisaged that the temperature probe will be interfaced with an electronic controller (9) that can actuate heating and cooling of the vessels. Temperature control can be carried out via the rigid container vessel that supports the bioreactor. This container would heat and cool the bioreactors by means of a water jacket or electrical heating & cooling system. A disposable heat exchanger could also be placed in the circulation loop, but this may circulation flow rate in the recycle loop.

In one embodiment, the rigid body used to support the bioreactor bag (1) is constructed to form a hollow jacket. A heat transfer liquid circulating through the rigid jacket is used to adjust the heating and cooling of the bioreactor container by actuating heaters and coolers located on the jacket circuit. Alternatively, a heat transfer liquid circulates through an outer lining of the bioreactor container to adjust the temperature of the bioreactor by actuating heaters and coolers located on the rigid support jacket circuit. In a further embodiment, an electrical heating blanket attached to the rigid support jacket is used to adjust the temperature of the bioreactor apparatus. Cooling of the bioreactor is either through natural heat loss or through an indwelling cooling finger positioned in a pocket in the bioreactor. A heat transfer fluid is passed through the cooling finger when required to adjust the temperature of the bioreactor.

Liquid additions need to be made to the bioreactor for inoculation of the bioreactor apparatus with cells, to control addition of acids and alkali, and to add additional nutrients to aid cell growth or remove reactor contents for further processing. These liquid additions may be in presterilized bags similar in composition to the bioreactor container material (for example PVC) or any other feed vessel used by those skilled in the art.

In one embodiment a source (such as a container) of liquid feed is aseptically connected to a feed line on the bioreactor apparatus and the contents of the source transferred into the bioreactor immediately. This would be used to add pre-sterilized liquid nutrients to the vessel before or after inoculation with cells. This route would also be used to add cell containing inoculum to the bioreactor.

In another embodiment, the addition of feed of a liquid is controlled by the action of a pinch valve. When the pinch valve is open, liquid flows into the bioreactor under the action of gravity or pressure applied to the container. When the pinch valve is closed, no feed flows into the bioreactor.

In a further embodiment, the addition of liquid feed is controlled by the action of a peristaltic pump. The feed tube connecting the feed container to the bioreactor container is placed into the peristaltic pump. When the peristaltic pump is on, liquid flows into the bioreactor apparatus at a constant rate using the peristaltic action of the pump. When the pump is off, no feed liquid flows into the bioreactor. The pump feed rate can be adjusted electronically to control the feed addition rate to the bioreactor.

To achieve optimal growth of culture the pH of the culture fluid in the bioreactor apparatus must be controlled within a tight operating range. The pH may be sensed using a pH probe located within the bioreactor container in an indwelling sheath, or as shown in FIG. 1, with a pH probe (11) within the circulation system, or as an integral part of the bioreactor container. The pH probe may be an electrochemical probe or an optical based sensor that uses spectrographic or fluorescence measurements to infer culture fluid pH. The pH probe may be interfaced with an electronic controller that can actuate addition of acid or alkali solutions into the bioreactor thus controlling the bioreactor pH to be maintained at the operator specified set point.

In one embodiment, conventional electrochemical pH probes, may be housed in T-fittings and located in-line on the circulation system. The pH may be then controlled via peristaltic pumps from the feed line directly linked to the bioreactor container.

In another embodiment, spectroscopic or fluorescent patch based chemical sensors can be positioned inside of the bioreactor container or circulation system. An LED based emitter and detector is then located next to the patch, external to the bioreactor apparatus. The LED emitter detector senses changes in the spectroscopic and/or fluorescent properties of a pH reactive dye immobilised in the patch. The spectroscopic and/or fluorescent signal is directly proportional to the bioreactor pH.

In addition to these pH sensor embodiments, two type of actuators can be used to add acid and alkali solutions that are used to control pH. In one embodiment, acid and alkali are fed into the vessel by actuating a peristaltic pump which induces flow through flexible tubing connecting the acid/alkali reservoir to the bioreactor. The reservoirs can be constructed of similar material to the bioreactor or glass bottles can be used as reservoirs and sterile connected to the bioreactor prior to operation

To achieve optimal growth of culture the dissolved oxygen tension (DOT) of the culture fluid in the bioreactor apparatus must be controlled within a tight operating range. It is envisaged that the DOT can be sensed using a DOT probe located within the bioreactor container in an indwelling sheath, or as shown in FIG. 1, with a DOT probe (12) in the circulation system loop, or as an integral part of the bioreactor container. The DOT probe may be an electrochemical probe or optical based sensor that uses spectrographic or fluorescence measurements to infer culture fluid DOT. The DOT probe could be interfaced with an electronic controller that can actuate increased liquid jet flow rate, inlet gas flow rate or inlet gas composition into the bioreactor thus controlling the bioreactor DOT to be maintained at the operator specified set point.

In one embodiment, conventional electrochemical DOT probes are housed in T-fittings and located in-line on the circulation system. The DOT probes are linked to a controller unit such that control can then be actuated by increased liquid jet flow rate, inlet gas flow rate or inlet gas composition into the bioreactor.

In a further embodiment, spectroscopic or fluorescent patch based chemical sensors are positioned inside of the bioreactor container or circulation loop. An LED based emitter and detector is located next to the patch, external to the bioreactor. The LED emitter detector senses changes in the spectroscopic and/or fluorescent properties of a DOT reactive dye immobilised in the patch. The spectroscopic and/or fluorescent signal is directly proportional to the bioreactor DOT. The DOT probes are linked to a controller unit such that control can then be actuated by increased liquid jet flow rate, inlet gas flow rate or inlet gas composition into the bioreactor.

The bioreactor of the present invention maybe used to culture cells (e.g. eukaryotic cells such as mammalian cells) and/or micro-organisms (such as E. Coli). Typically such micro-organisms and cells are cultured to obtain a product of interest such as a polynucleotide (e.g. plasmid DNA), polypeptide, or protein (such as a therapeutically useful protein e.g. antibody or antibody fragment). Examples of mammalian cells are host cells such as CHO, NSO, COS, BHK, Y2/0 transformed or transfected with a vector comprising a polynucleotide encoding a protein or polypeptide of interest. Such techniques for the production of a protein or polypeptide of interest are well known to those skilled in the art. Typically such host cells are cultured in serum-free culture.

The following are intended as non-limiting examples of the invention. The specific embodiments described within the examples may be modified as set forth in the claims.

### EXAMPLE 1

Identification of Design Parameters in a Scale Down Model Venturi Plunging Jet Bioreactor

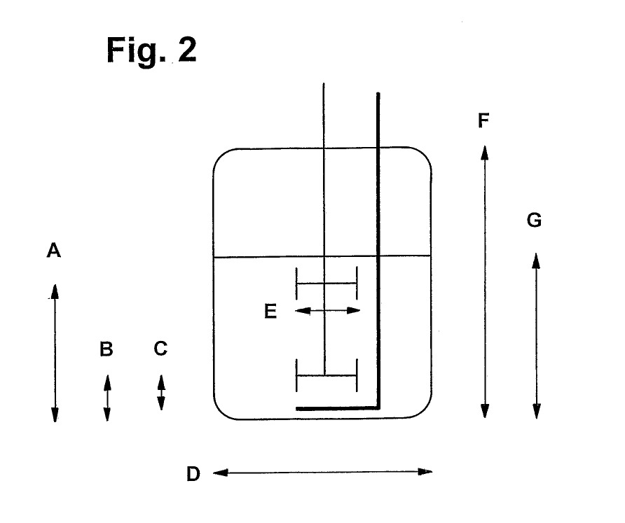

To guide the design of a disposable reactor, a conventional non-disposable reactor was configured to operate in both stirred tank and disposable mode to allow direct comparison of the two culture systems. A water jacketed Applikon bioreactor with a total volume of 7 L was used in all design studies. The headplate was fitted with two venturi jets and an external circulation loop. The bioreactor contents were pumped from the vessel using a Quattro diaphragm pump via a wide bore harvest tube circulated back to the venturi jets in a similar manner as shown in FIG. 1. The dimensions of the venturi jets (illustrated in FIG. 3) were detailed above. The jet flow rates were between 5 and 9 liters per minute (equivalent to 2.95 and 5.3 meters/sec). The bioreactor was also equipped with baffles, pH probe (Ingold) and polarographic DOT probe (Mettler Toledo) and a PT100 Temperature probe inserted into a temperature pocket. The dimensions of the test bioreactor (illustrated in FIG. 2) are shown below.

| | Measurement | (cm or L) |
|---|---|---|
| | External diameter on headplate | 19.0 |
| | Vessel Total volume | 7.0 L |
| | Vessel max working volume | 5.0 L |
| A | Top Impeller height from base | 11.5 |
| B | Bottom Impeller height from base | 6.0 |
| C | Distance between bottom impeller and | 3.0 |
| | sparge | |
| D | Vessel internal diameter | 16.0 |
| E | Impeller diameter | 5.3 |
| F | Total vessel height | 35.0 |
| G | Maximum ungassed liquid height | 18.0 |

Notes:
Two 6 bladed Ruston impellers are installed on agitator according to the distances indicated above. Impeller heights are set such that the top impeller is below the ungassed liquid height.

All probes were connected to an Applikon 1030 biocontroller. Temperature and agitation speed were controlled from the Applikon 1035 bioconsole.

Bioreactor Setup

An Applikon based benchtop bioreactor system with a total volume of 7 litre was used in this study (see FIG. 2). The physical characteristics of the test bioreactor are indicated in FIG. 2 with reference to the dimensions given above. The bioreactor was also fitted with three baffles, thermometer pocket, pH probe, a DOT probe, a sampling tube, a multipoint gas sparge line and a harvest line. Two venturi jets (see FIG. 3) were fitted on opposite sides of the headplate and connected to an external circulation loop. The bioreactor contents were recirculated from the harvest line into the venturi plunging jets via a Quattro diaphragm pump (Pall corporation) on an external circulation loop. An agitator with two ruston type impellors are located in the center of the vessel to allow comparison of results between a conventional stirred tank and venturi plunging jet system. Gas mixing measurements were carried out with 4 L of reverse osmosis (RO) water in the vessel maintained at 37° C. by means of a circulating water jacket on the bioreactor.

Dissolved O₂ Calibration and Data Acquisition

The dissolved O₂ tension (DOT) in the bioreactor was measured using an in-situ polarographic DOT probe located in the vessel. This was connected to an Applikon 1035 biocontroller which was configured to output the DOT value to analog output 1. The probe was calibrated to 0% using a DOT simulator and to 100% after the bioreactor had been sparged for 15 min at 5 L/min with compressed air at an agitator speed of 800 rpm. DOT=0.0% and 100% gave a reading of 4.0 mA and 20 mA, respectively at the analog output. A Picologger ADC16 analog to digital converter (Pico Technologies) was used to collect readings every 500 msec, which were transferred to a PC in real time over an RS232 link. The 4-20 mA signal from the Applicon 1035 was converted to 0-2 V signal, suitable for the ADC16 using a suitable signal conversion card.

Venturi Plunging Jet Design Parameters Identification by Response Surface Methodology

StatEase Design Expert 6.0 software was used for all design setup and analysis. A Box-Behnken response surface design was used to investigate the relative effects of each of the parameters. Four factors were considered in the design, Gas flow rate, Liquid flow rate, Venturi ratio and jet height. The design was separated into 3 blocks to take into account day to day variation.

Table 2 provides a summary of the response surface design set up. The gas mixing efficiency of the bioreactor operated in conventional mode was also determined each day to ensure block to block consistency. The conventional runs were carried out at 37° C., 4 L working volume, compressed air at 4 L/min and agitator speed at 700 rpm.

Gas Mixing in Conventional Stirred and Sparged Tank Bioreactors: External Controls and Operating Range

The gas mixing efficiency of the bioreactor operated in a conventional mode was investigated to provide a comparison with the jet mixing results. In a conventional microbial run, gas flow and agitator are set to 2 L/min and 500 rpm. Under normal run conditions the agitator speed is automatically controlled between 500 and 900 rpm and the gas flow rate controlled between 2 L/min and 8 L/min. For compressed air flow rates at 2 L/min and agitator speeds of 500, 700 and 900 rpm, the K_{L}a were 0.019, 0.033 and 0.046 sec⁻¹ respectively. External control measurements of K_{L}a were also carried out at the start of each experiment block. These external controls were carried out at 4 L/min at 700 rpm.

TABLE 2
Design summary of factors and responses.

| | Range (units) | Note |
|---|---|---|
| Factors | | |
| A-gas Flow rate | 1 to 3 (L/min) | Gas Flow rate though the |
| | | venturi nozzle |
| B-Liquid Flow rate | 5 to 9 (L/min) | Liquid flow rate through the |
| | | circulation loop |
| C-Jet Height | 1 to 5 (cm) | Height the jet exit above the |
| | | ungassed liquid surface |
| D-Venturi ratio | 0.2 to 0.8 | Ratio of distance of venturi |
| | | nozzle is between the liquid |
| | | entry point and the liquid jet |
| | | nozzle |
| Responses | | |
| K_{L}a | (s⁻¹) | A measure of the gas |
| | | mixing efficiency. K_{L} is a |
| | | measure of the gas transfer |
| | | rate and a is the interfacial |
| | | area available for gas |
| | | transfer. |
| Plunge Depth | (cm) | Distance between the |
| | | ungassed liquid height and |
| | | the average jet plunge |
| | | depth |

The DOE was separated into 3 blocks to take into account day to day variation.
For liquid flow rates of 5, 7 & 9 L/min, the pump was set to 658, 929 and 1200 rpm respectively.

Results

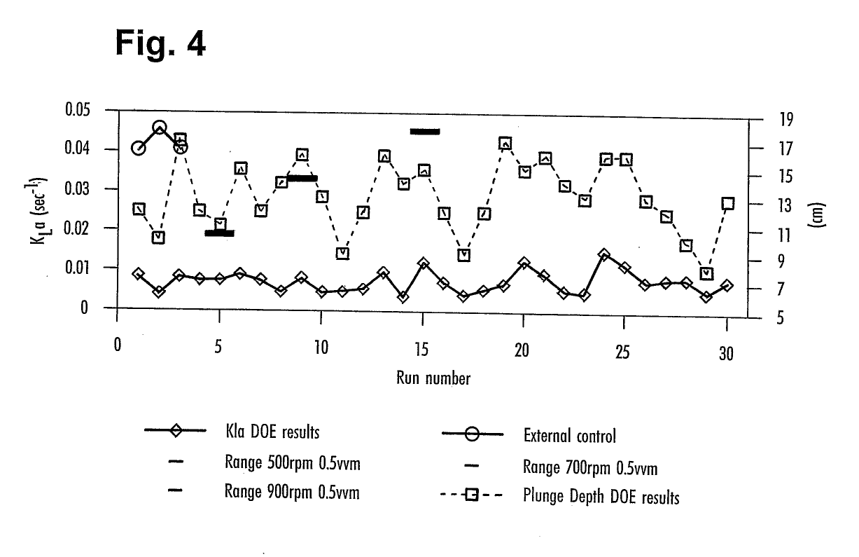

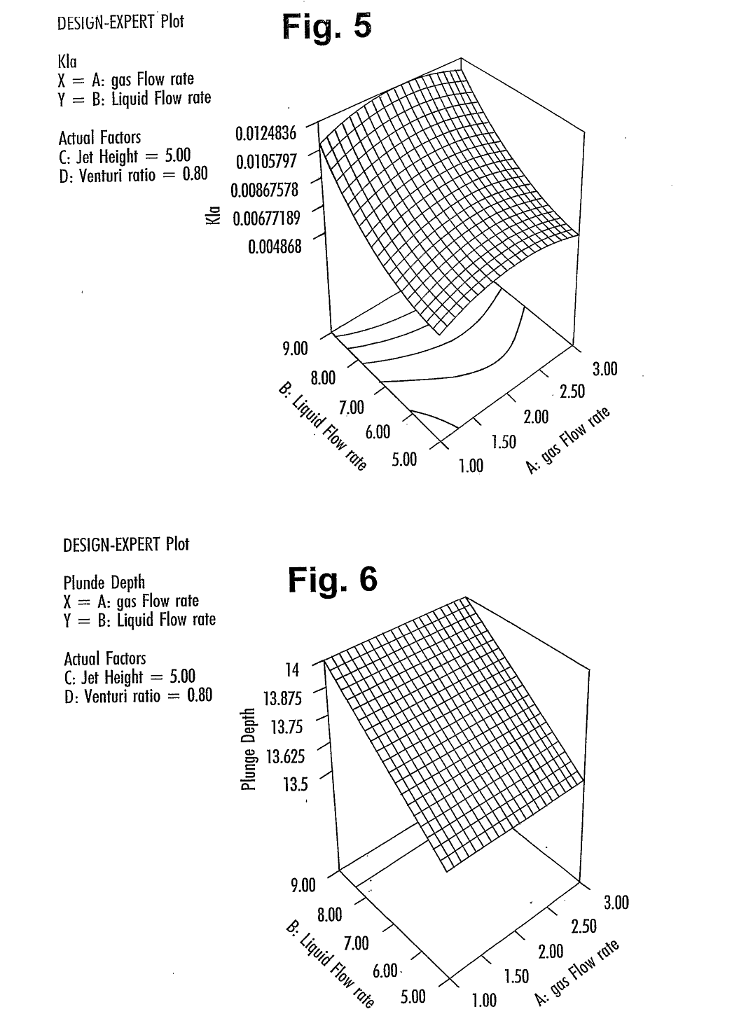

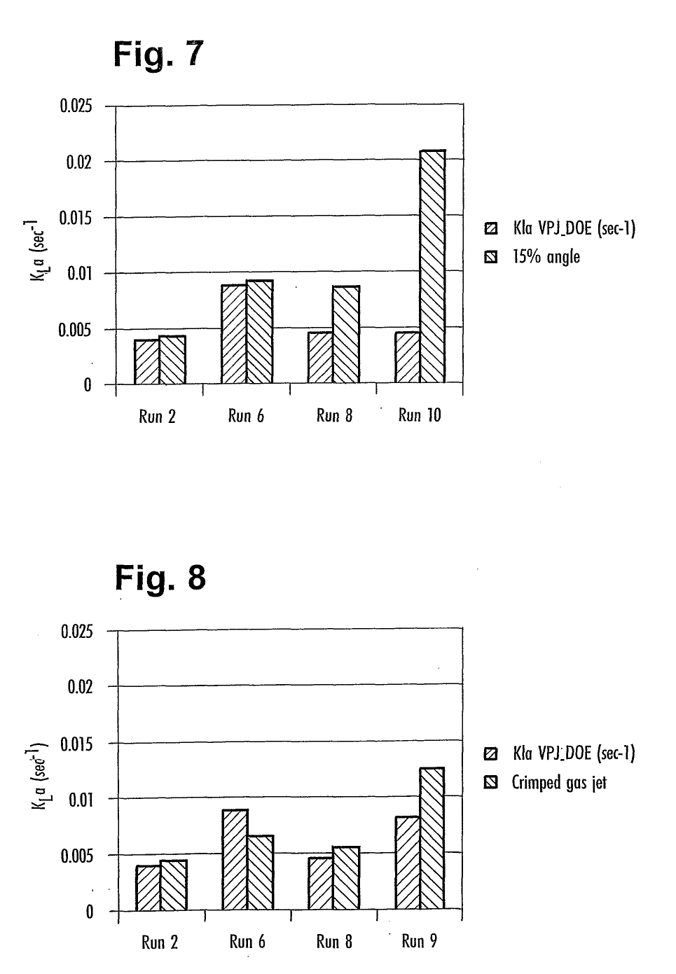

FIG. 4 shows a summary of the K_{L}a and plunge depth results obtained from the initial response surface design. The operating range and external controls are also indicated. The K_{L}a values from the design, range from 8% to 35% of the external controls. A summary of the analysis is shown in Table 3. Liquid flow rate and Venturi ratio are the main parameters affecting K_{L}a, while Gas flow and Liquid flow contributes a small non-linear effect. Many reports have identified jet height as an important factor contributing to jet mixing (reviewed in Bin, 1993, Chem. Eng. Sci, 48: 3585-3630). The model used indicates that jet height had no significant effect at this scale but it is expected that the gas mixing performance will improve as the vessel size and jet height is increased. Venturi ratio would not be an obvious main effect as the difference in gas contact time at different venturi ratios would be considered as not significant.

Table 3 identifies that liquid flow rate and venturi ratio are the main parameters affecting plunge depth, while the effect of jet height was not considered to be significant. Interactions between liquid flow and venturi ratio and jet height and venturi ratio were identified as having a significant effect on plunge depth (Table 3). The squared terms of liquid flow rate and gas flow rate were noted to have a significant effect in the K_{L}a response.

TABLE 3
Summary of model output terms identified by Design Expert 6 software

| | K_{L}a Model | Plunge Depth Model |
|---|---|---|
| Model Terms | Prob > F | Prob > F |
| A: Gas Flow | 0.4843 | Excluded |
| B: Liquid Flow | <0.0001 | <0.0001 |
| C: Jet Height | 0.0677 | 1.0000 |
| D: Venturi Ratio | 0.0016 | 0.0021 |
| A²: (Gas Flow)² | 0.0418 | Excluded |
| B²: (Liquid Flow)² | 0.0199 | Excluded |
| C²: (Jet Height)² | Excluded | Excluded |
| D²: (Venturi Ratio)² | Excluded | 0.0164 |
| BD | Excluded | 0.0137 |
| CD | Excluded | 0.0283 |

Note.
Terms with Prob > F greater than 0.1 that do not have significant higher order terms have been excluded automatically. Significant terms are indicated in bold.

Additional Design Modifications
Jet Angle

Improved lateral mixing can be achieved by changing the angle at which the jets hit the liquid surface. Tojo et al (Tojo, Naruko and Miyanami, 1982, Chem. Eng. J., 25:107-109) identified that angles between 15 and 20° had a significant effect on gas mixing. FIG. 1 shows the effect of placing a 15° angle at the jet exit on K_{L}a. Experiments were carried out at four design points. Although no significant effects were observed at low gas and liquid flow rate (run 2 and run 6), K_{L}a was increased 4 fold over the non-angled jet at high gas and liquid flow rates (run 10).

Crimped Gas Outlet in the Venturi

The interfacial area between the liquid jet and the gas jet exiting the venturi can be increased by crimping the gas outlet. The effect of a crimped ventur gas inlet was insignificant at low gas and liquid flow rates (FIG. 8: Run 2 and 6). Some minor increases in K_{L}a were observed at high gas and liquid flow rate combinations (FIG. 8: Run 8 and 9).

### EXAMPLE 2

Optimisation of Design Parameters in a Scale Down Model Venturi Plunging Jet Bioreactor

Example 1 identified the venturi ratio, jet angle and to a more limited extent jet crimpling as important design parameters that affect the performance of a venturi plunging jet-based bioreactor. Although jet height was not identified as an important parameter, jets were set to the highest possible position (i.e. the highest possible position that can ensure angled entry of the jet into the culture fluid). Bin (Bin, A. K., 1993, Chem. Eng. Sci. 48 (21): 3585-3630) identified jet height as an important design parameter for plunging jets. In the single use bag design, this would prevent damage to the bag integrity during packaging and transportation. In this example a response surface design approach is used characterise the performance of the venturi jet in response to gas flow rate, liquid flow rate and venturi ratio where jet height was set high. The jets were fitted with 15° angles and a crimped gas tube and the 40% Oxygen was used as the inlet gas.

Bioreactor Setup

The test bioreactor was the same set up as used in Example 1. Jet flow rates were between 2 and 9 liters/minute, equivalent jet velocities of 1.18 and 5.3 meters/second. The gas flow rates for example 1 and 2 are between 1 litre and 9 litre/minute.

Dissolved O₂ Calibration and Data Acquisition

The same calibration procedure used in example 1 was used to set up the Dissolved Oxygen tension (DOT). In this example the 1035 applikon biocontroller was set to output 4 mA for 0% DOT and 20 mA for 150% DOT. The setting on the picologger analog to digital converter were the same as described in Example 1.

Venturi Plunging Jet Design parameters identification by Response Surface methodologyStatEase Design Expert 6.0 software was used for all design setup and analysis. A Box-Behnken response surface design was used to investigate the relative effects of each of the parameters. Three factors were considered in the design, Gas flow rate, Liquid flow rate and Venturi ratio. The design was separated into 3 blocks to take into account day to day variation. provides a summary of the response surface design set up. The gas mixing efficiency of the bioreactor operated in conventional mode was also determined each day to ensure block to block consistency. The conventional runs were carried out at 37° C., 4 L working volume, compressed air at 4 L/min and agitator speed at 700 rpm.

TABLE 4
Design summary of factors and responses.

| | Range (units) | Note |
|---|---|---|
| Factors | | |
| A-gas Flow rate | 0.55 to 9.0 (L/min) | Gas Flow rate though the |
| | | venturi nozzle |
| B-Liquid Flow | 0.55 to 10.45 (L/min) | Liquid flow rate through the |
| rate | | circulation loop |
| C-Venturi ratio | 0.2 to 0.8 | Ratio of distance of venturi |
| | | nozzle is between the liquid |
| | | entry point and the liquid jet |
| | | nozzle |
| Responses | | |
| K_{L}a | (s⁻¹) | A measure of the gas |
| | | mixing efficiency. K_{L} is a |
| | | measure of the gas transfer |
| | | rate and a is the interfacial |
| | | area available for gas |
| | | transfer. |
| Plunge Depth | (cm) | Distance between the |
| | | ungassed liquid height and |
| | | the average jet plunge |
| | | depth |

The DOE was separated into 3 blocks to take into account day to day variation.

Results

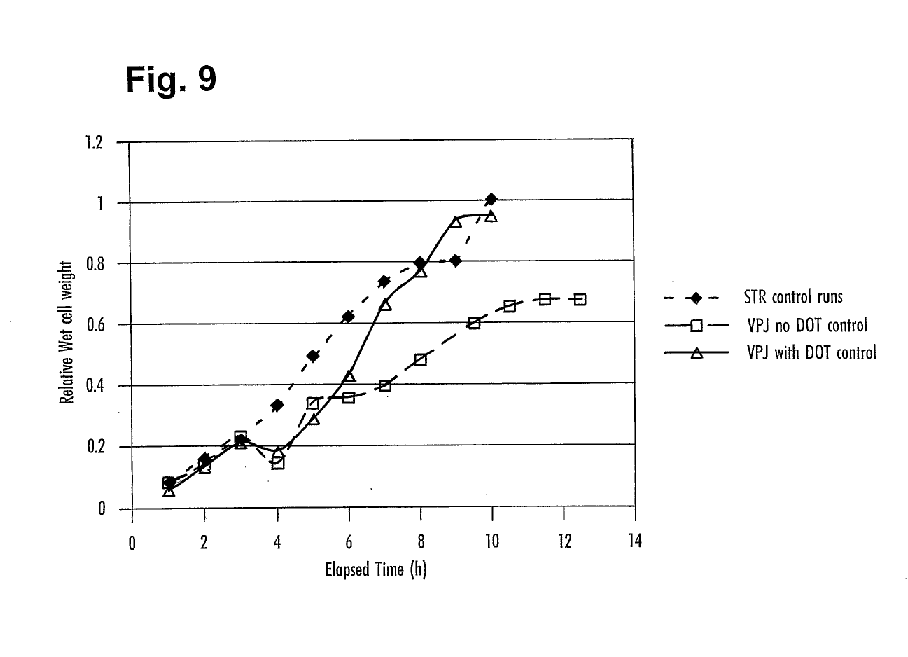

FIG. 9 shows a summary of the K_{L}a and plunge depth results obtained from the example 2 response surface design. Table 5 shows a summary of the response surface analysis and as in example 1 identifies gas flow rate and liquid flow rate as critical parameters. Interactrions between gas flow rate and liquid flow rate and venturi ratio are also identified as critical.

The operating range and external controls are also indicated. The K_{L}a values from the design range from 5% to 98% of the external controls (FIG. 9). This demonstrates that the modified venturi plunging jet design is capable of acheiving gas exchange rates similar to mixing rates in conventional stirred tank bioreactor.

Gas Flow rate and Liquid flow were also identified as the main parameters affecting plunge depth. Plunge depth is also non-linear function of liquid flow (B²). No significant interactions between liquid flow, venturi ratio were identified as having a significant effect on plunge depth.

TABLE 5
Summary of model output terms identified by Design Expert 6 software

| | K_{L}a Model | Plunge Depth Model |
|---|---|---|
| Model Terms | Prob > F | Prob > F |
| A: Gas Flow | <0.0001 | 0.0488 |
| B: Liquid Flow | <0.0001 | <0.0001 |
| C: Venturi Ratio | 0.6737 | Excluded |
| A² (Gas Flow)² | Excluded | Excluded |
| B² (Liquid Flow)² | Excluded | <0.0001 |
| C²: (Venturi Ratio)² | Excluded | Excluded |
| AB | 0.0006 | Excluded |
| AC | 0.0067 | Excluded |

Note.
Terms with Prob > F greater than 0.1 that don't have significant higher order terms have been excluded automatically. Significant terms are indicated in bold.

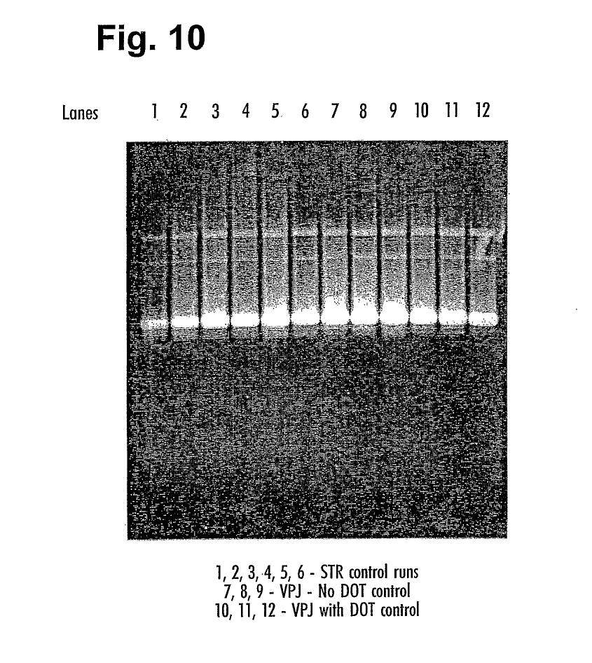

FIG. 10 shows the predicted effect of varying the venturi ratio on oxygen transfer. At low venturi ratio, increased gas flow rate have a detrimental effect on KLa. At high venturi ratio, the dynamic range of achievable K_{L}a is increased and increasing gas flow rate has a positive effect on K_{L}a.

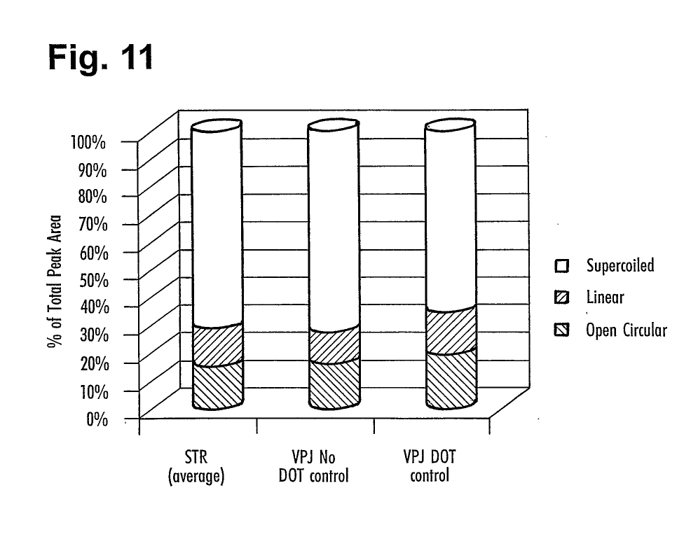

When the bioreactor is operated in conventional stirred tank mode, K_{L}A between 0.01 and 0.04 s⁻¹ were achieved (FIG. 4 and FIG. 9: Conventional STR). FIG. 11 shows response surface model predictions for gas and liquid flow rates required to achieve a minimum K_{L}A of 0.01 s⁻¹ and shows that the Venturi plunging jet bioreactor is capable of supporting oxygen transfer requirements for microbial and cell culture.

### EXAMPLE 3

Comparison of Small Scale Model Venturi Plunging Jet Operation to Venturi Plunging Jets for a 50 L Working Volume Single Use Bioreactor

In this example, the small scale venturi plunging jet was scaled up to the dimensions for a pilot scale disposable bioreactor with a working volume of 50 L. The liquid jet velocity exiting the pilot scale were kept in the same range as the liquid velocities used in Example 1 and example 2. The dimensions for the scaled up jet are shown in FIG. 3.

Two large scale jets with 15° jet outlet angles were fixed to an opened topped 100 L vessel containing water at 25° C. Oxygen transfer rates were measured using the same DOT probe set up as in example 1 and 2. Jets were set at low medium and high jet height. (10, 20 and 30 cm above the ungassed liquid height). FIG. 12 compares the K_{L}a predicted from the small scale model to the K_{L}a obtained from the scaled up jets. The results from the small scale and scaled up venturi plunging jets are in good agreement and show improved gas mixing performance as the jet hight increases.

### EXAMPLE 4

Growth comparison of a recombinant Escherichia coli DH1 in a scale down venturi plunging jet bioreactor.

The bioreactor configured as described in Example 1 was used to compare the growth characteristics of a recombinant E. coli grown in a conventional stirred tank reactor (STR) and venturi plunging jet reactor (VPJ) mode. The E. coli had been transformed with a pUC based plasmid, pXY, used as a DNA based therapeutic vaccine.

Methods

Biomass Concentration Measurement Methods

Optical Density (OD) Measurement

Optical density was measured using a Pharmacia NovaSpec spectrophotometers set at 600 nm. Samples were diluted in sterile medium to give a reading between 0.2 and 0.7. The OD of the culture was calculated by multiplying the reading by the dilution factor.

Wet Cell Weight (WCW) Measurement

Aliquots (1 mL) of culture samples were transferred into duplicate preweighed 2.2 mL microcentrifuge tubes. Tubes were centrifuges for at room temperature for 10 mins at 14000 g in an Eppendorf 5471 microcentrifuge. The culture supernatant was poured out and residual liquid removed with a cotton bud. The tubes were then reweighed. The weight difference in the tubes was used to calculate the Wet cell weight concentration.

Growth Medium

Seed and expansion flask growth medium used in this study was single strength Terrific Broth. Double strength Terrific broth was used in Bioreactor growth studies.

Inoculum Preparation

Frozen stock cultures of the transformed E. coli DH1 transformed with pXY plasmid therapeutic vaccine were maintained in 15% (v/v) glycerol stocks stored at −70° C. These glycerol stocks were revived into 500 mL baffled shake flasks containing 100 mLs of seed medium (with 50 mg/L Kanamycin). The revival flask was incubated at 37° C. for 8 hours at 230 rpm. After 8 hours, the culture was expanded into three 500 mL baffled shake flasks containing seed medium and Kanamycin. Expansion flasks were inoculated to an Optical density ( at 600 nm)=0.02 and incubated for 16 hours at 37° C. and 230 rpm. The contents of the three shake flasks were pooled and optical density read prior to inoculation. The volume of inoculum transferred in the bioreactor was set to achieve an initial starting OD=0.4.

Stirred Tank Bioreactor (STR) Operation

The temperature was controlled at 37° C. using a temperature controlled water jacket on the vessel. Automatic addition of alkali (2M Sodium hydroxide) and acid (2M Sulphuric acid) was used to maintain the pH at 7.0. For operation in both STR and VPJ mode, the DOT probe was calibrated at 100% saturation by running the bioreactor in STR mode, setting the agitator to 700 rpm and sparging compressed air at 2 vvm through the sparge line for 15 min. In STR mode, Dissolved oxygen tension (DOT) in the STR was maintained above 30% by automatic cascade control of agitator speed and gas flow rate. When the controller was no longer able to maintain DOT >30%, the inlet gas supply was manually changed to oxygen enriched air, containing 40% O2 in Nitrogen.

Venturi Plunging Jet (VPJ) Operation

Temperature and pH were maintained as described for STR operation. DOT was controlled manually by modifying the liquid flow rate and gas flow rate. Both VPJ runs were carried out with a venturi ratio of 0.8 and the jet height set to 5 cm. The jets were angled at 20° and fitted with crimped gas outlets. The initial liquid and gas flow rates were 4 L/min and 2 L/min respectively. For the VPJ run without DOT control, liquid flow rate and gas flow rate were changed every 2 hrs to maintain DOT above 30%. The liquid flow and gas flow rate were change every 0.5 hours to maintain DOT above 30% for the VPJ run with DOT control.

Results

Time course profiles of the evolution of wet cell weight is shown in FIG. 13. The results from duplicate stirred tank bioreactor were averages and all results are plotted relative to the maximum average wet cell weight attain in the stirred tank bioreactor. Growth in the VPJ bioreactor without DOT control (open squares) was initially similar to the growth in the STR but slowed up after 3 hours due to oxygen limitation. Final wet cell weights attained in the VPJ bioreactor without DOT control were 70% of those attained in the control stirred tank bioreactors. Growth in the VPJ with DOT control followed a similar pattern to the VPJ without DOT control for the first 4 hours. Final wet cell weight concentration in the VPJ with DOT control after 8 hours was not significantly different from the wet cell weights attained in the STR bioreactors. Table 6 shows the growth rates relative to the average growth rates attained during different growth phases in the stirred tank bioreactor. Initial growth rates were calculated over the first 4 hours while final growth rates were calculated for the remaining duration that the culture was growing. The overall growth rate is calculated over the complete duration of the growth phase.

FIG. 14 shows an agarose gel loaded with plasmid DNA extracts prepared from bioreactors. Lanes 1-6 contain early middle and harvest samples from replicate stirred tank bioreactor fermentations. Lane 7-9 contain early middle and harvest samples derived from the venturi plunging jet reactor run without DOT control. Lane 10-12 contain early middle and harvest samples derived from the venturi plunging jet reactor run with DOT control product that was extracted using the alkaline lysis procedure. Densitometry analysis of the harvest samples and calculation of the relative peak areas (FIG. 15) show that there was no significant difference between the relative proportions of the plasmid DNA species in the plasmid extracts from different bioreactor configurations

TABLE 6
Growth rate comparison of E. coil DH1 pXY grown in conventional
stirred tank reactor and venturi plunging jet reactors.

| | | | Overall growth |
|---|---|---|---|
| | Initial growth rate | Final Growth rate | rate |
| Average | 1.00 | 1.00 | 1.00 |
| VPJ without | 0.41 | 1.00 | 0.71 |
| DOT control | | | |
| VPJ with | 0.81 | 1.20 | 0.92 |
| DOT control | | | |

Note:
Results are expressed relative to the maximum biomass achieved in the STR control runs (N = 2)

1. A bioreactor apparatus for culturing micro-organisms or cells in a culture fluid comprising a container for the culture fluid and a circulation system to circulate the culture fluid out of, and back into, the container, wherein the circulation system has a mechanism adapted in use to form the culture fluid into a hollow flow stream and to introduce an oxygen-containing gas stream into the hollow of the flow stream of the culture fluid.

2. The apparatus of claim 1 wherein the circulation system has a mechanism adapted in use to form the culture fluid into an annular flow stream having a hollow core and to introduce an oxygen-containing gas stream into the hollow core of the annular flow stream of the culture fluid.

3. The apparatus of claim 1 or claim 2, wherein the mechanism comprises at least one pair of inner- and outer-arranged tubes, the inner tube of the at least one pair being in flow communication with a supply of the oxygen-containing gas and the outer tube being in flow communication with the container whereby the culture fluid is able to flow in the outer tube over the inner tube to form the hollow flow stream into the hollow of which the oxygen-containing gas is able to be introduced via the inner tube.

4. The apparatus of claim 3 wherein the inner- and outer-arranged tubes are concentrically arranged.

5. The apparatus of any preceding claim, wherein the circulation system has at least one efflux nozzle with an outlet oriented into the container for delivering the culture fluid back into the container.

6. The apparatus of any one of claims 2 to 5, wherein the end of the at least one pair of inner- and outer-arranged tubes is configured as the efflux nozzle, optionally having an angled tip section.

7. The apparatus of any one of claims 2 to 6, wherein the outer and inner tubes each have an outlet and the inner tube outlet is disposed inside the outer tube.

8. The apparatus of claim 7 when appended to claim 6, wherein the outlet of the outer tube is in an angled tip section of the outer tube.

9. The apparatus of claim 8, wherein the angled tip section has an upstream end which extends to the outlet in a downstream direction from a point on the outer tube which is level with, or downstream of, the outlet of the inner tube.

10. The apparatus of claim 6 or any other claim appended thereto, wherein the outlet of the outer tube is the outlet of the efflux nozzle.

11. The apparatus of claim 5 or any claim when appended thereto, wherein the container has a bottom wall and a sidewall structure upstanding from the bottom wall and wherein the outlet of the at least one efflux nozzle is spaced above, and oriented in the direction of, the bottom wall.

12. The apparatus of claim 11, wherein the outlet of the at least one efflux nozzle is oriented in the direction of the sidewall.

13. The apparatus of claim 12, wherein there are two or more efflux nozzles, the outlets of which all facing in the same rotary sense about an upward-downward axis of the container.

14. The apparatus of any one of the preceding claims in which the mechanism is adapted such that the oxygen-containing gas is entrained into the hollow of the culture fluid flow stream.

15. The apparatus of claim 3 or any other claim appended thereto, wherein the outer and inner tubes are arranged such that the oxygen-containing gas is able to be drawn into the hollow of the flow stream in the outer tube by the venturi effect.

16. The apparatus of any one of the preceding claims in which one or more parts are designed for single use.

17. The apparatus of any one of the preceding claims in which the container for the culture fluid is constructed from a waterproof semiflexible or flexible material.

18. The apparatus as claimed in claim 17 wherein the container for the culture fluid is constructed from polyvinyl chloride, or one or more layers of PVC or PTFE sheets.

19. The apparatus of any one of the preceding claims in which the circulation system is constructed of waterproof semiflexible or flexible material.

20. The apparatus as claimed in claim 19 wherein the circulation system is constructed of silicon elastomer or platimum treated silicon elastomer.

21. The apparatus of any one of the preceding claims in which the circulation system has a pump for pumping the culture fluid out of the container and back there into.

22. The apparatus as claimed in claim 21 wherein a peristaltic pump is used in which the pump heads do not come into direct contact with the culture fluid.

23. The apparatus as claimed in claim 21 wherein a pump with a disposable pump head is used.

24. The apparatus of any one of the preceding claims which further comprises sensors in order to optimise the control of temperature, and/or pH and/or dissolved oxygen tension.

25. The apparatus as claimed in claim 24 wherein the sensors are located within the circulation system.

26. The apparatus as claimed in claim 24 wherein the sensors are located within the container.

27. A process for culturing micro-organisms or cells in a culture fluid in which the culture fluid is circulated out of and back into a reservoir of the culture fluid, wherein during the circulation step the culture fluid is formed into a hollow flow stream and an oxygen-containing gas is introduced into the hollow of the flow stream.

28. The process of claim 27, wherein there is a continuous circulation of the culture fluid.

29. The process of claim 27 or 28, wherein the culture fluid is circulated with the micro-organisms or cells therewithin.

30. The process of claim 27, 28 or 29, wherein the culture fluid is jetted back into the reservoir.

31. The process of claim 30, in which the culture fluid is jetted back into the reservoir from above the reservoir surface.

32. The process of claim 30 or 31 in which the culture fluid is jetted back into the reservoir such as to produce rotation of the reservoir about an imaginary axis passing through the surface of the reservoir.

33. A process for culturing micro-organisms or cells in a culture fluid in which the culture fluid is removed from a reservoir of the culture fluid and jetted back into the reservoir surface to produce rotation of the reservoir about an imaginary axis passing through the reservoir surface.

34. A bioreactor apparatus for culturing micro-organisms or cells (such as mammalian, yeast or plant cells) in a liquid culture fluid comprising a culturing container for culturing said micro-organisms or cells within the culture fluid which apparatus comprises a circulation system to circulate the culture fluid out of, and back into, said culturing container wherein the circulation system comprises at least two pairs of concentrically arranged outer and inner tubes whereby the inner tube of said at least two pairs being in fluid communication with a supply of oxygen-containing gas and the outer tube being in flow communication with the culturing container such that the culture fluid is able to flow in the outer tube over the inner tube to form a hollow flow stream into the hollow of which the oxygen-containing gas is able to be introduced via the inner tube, said at least two pairs comprising an efflux nozzle from where said oxygen-containing hollow flow stream fluxes out of said at least two pairs and into said culturing container.

35. The apparatus of claim 34 wherein at least one of said at least two pairs are spaced above the liquid culture fluid.

36. The apparatus of claim 34 or 35 wherein at least one of the efflux nozzles is spaced above the liquid culture fluid and orientated to flux said oxygen-containing hollow flow stream into said culturing container, preferably orientated to flux said flow stream into said liquid culture fluid.

37. The apparatus of claim 36 wherein the efflux nozzle is configured to form said oxygen-containing hollow flow stream into a jet.

38. The apparatus of any one of claims 34 to 37 wherein the apparatus comprises two, three or four pairs of concentrically arranged outer and inner tubes.

39. The apparatus of any one of claims 36 to 38 wherein the efflux nozzle is orientated to flux said flow stream into said liquid culture fluid at an inclined angle of 70 to 75° or thereabout to the plane of the liquid culture fluid.

40. The apparatus of claim 34 wherein the oxygen-containing hollow flow stream of at least one of said at least two pairs fluxes out of said nozzle at an angle of between 15° to 20° from a longitudinal axis of said outer tube from which said nozzle depends.

41. The apparatus of claim 40 wherein the oxygen-containing hollow flow stream of both or all of said at least two pairs fluxes out of said nozzle at an angle of between 15° to 20° from a longitudinal axis of said outer tube from which said nozzle depends.

42. The apparatus of any preceding claim wherein the venturi ratio is between 0.2 to 0.8 inclusively, preferably 0.5 or greater e.g. 0.6, 0.7 or 0.8.

43. A process for the production of a polynucleotide, polypeptide or protein of interest which process comprises culturing a micro-organism and/or cell in the culture fluid of the apparatus of any preceding claim.
